(19) United States
(12) Patent Application Publication
(10) Pub. No.: US 2026/0309687 A1
(43) Pub. Date: Oct. 8, 2026

(54) DEVICE AND METHOD FOR MONITORING GAS

(71) Applicant: PIOVAN S.P.A., Santa Maria di Sala, Veneto (IT)

(72) Inventors: Enrico BELLIO, Ponzano Veneto (IT); Davide CAPPELLINI, Soncino (IT)

(73) Assignee: PIOVAN S.P.A., Santa Maria di Sala, Veneto (IT)

(21) Appl. No.: 19/479,664

(22) PCT Filed: Jul. 8, 2024
(86) PCT No.: PCT/IB2024/056644

§ 371 Date: Oct. 28, 2025

(30) Foreign Application Priority Data
Jul. 14, 2023 (IT) 102023000014853

(51) Int. Cl. G01N 33/00; G01N 33/44

(52) U.S. Cl. CPC G01N 33/0026; G01N 33/0031; G01N 33/0047; G01N 33/442

(57) ABSTRACT

A monitoring device and a monitoring method are disclosed in which a monitoring chamber containing one or more olfactory sensors is supplied selectively with a process gas used in a deodorization process for deodorizing incoherent plastics, or with a base gas, for example ambient air, or with a cleaning gas, for example purified ambient air, using a first pump for conveying the process gas used along a first duct, a valve for connecting selectively a second duct to a source of the base gas or to a source of the cleaning gas, and a second pump for conveying the selected gas along the second duct.

### BACKGROUND OF THE INVENTION

The invention relates to a monitoring device and a monitoring method, in particular for monitoring odorous emissions, usable for example for providing indications of the trend of volatile organic carbon (VOC) and/or of total organic carbon (TOC) present in a gas used in an industrial process. The invention further relates to an apparatus for deodorizing incoherent plastics that uses the aforesaid monitoring device.

Specifically, but not exclusively, the invention can be advantageously applied for monitoring in real time a deodorization process for deodorizing incoherent plastics obtained at least partially from recycling, in particular of polyolefin, for example r_HDPE, r_PP, r_PE.

In particular, reference is made to a deodorization apparatus as in the preamble to claim 11. One example of this apparatus is disclosed in patent publication WO 2022/023905 A1.

The prior art further comprises various types of monitoring devices for monitoring odorous emissions. Patent publications WO 2020/144430 A1, WO 2015/068116 A1 and WO 2009/068965 A1 disclose some embodiments thereof.

Patent publication JP 2021523362 A discloses a monitoring device made according to the preamble of claim 1.

Various aspects of the prior art are improvable.

Firstly, particularly precise and reliable real-time monitoring of a deodorization process for deodorizing incoherent plastics is desirable.

Secondly, it is desirable to dispose of a monitoring device of relatively long duration which is at the same time constructionally simple and cheap.

Another improvable aspect is enabling real-time monitoring of a gas used in a deodorization process with a response that is as rapid as possible to any variation that may occur during the process.

### SUMMARY OF THE INVENTION

One object of the invention is to propose a solution that is able to overcome one or more of the aforesaid limits and drawbacks of the prior art.

One object of the invention is to provide an alternative device and/or an alternative apparatus and/or an alternative monitoring method to those of the prior art.

One object of the invention is to permit real-time monitoring of a deodorization process for deodorizing incoherent plastics, i.e. a process in which the incoherent plastics (for example, polyolefin) are traversed by a flow of a process gas (for example, air) that is heated and/or dehumidified to promote the extraction of odorous volatile substances from the material.

One advantage is to make an instrument available that is able to detect odorous substances emitted by incoherent plastics from recycling, in particular polyolefins like, for example, r_PE, r_PP, r_HDPE.

One advantage is to provide a monitoring device that enables the variation in the intensity of the odorous emissions in the flow of the process gas used in deodorization and sampled in situ by a hopper containing the incoherent plastics to be detected in real time by an olfactory sensor/by olfactory sensors.

One advantage is to permit particularly precise monitoring in real time of a deodorization process for deodorizing incoherent plastics.

One advantage is to devise a constructional simple and cheap monitoring device of relatively long duration.

One advantage is to give rise to a monitoring device that is able to operate in real time with a relatively very rapid response to any variation that may occur during a deodorization process.

One advantage is to procure a monitoring device that is easily integrable into a deodorization apparatus, in particular for monitoring a deodorization process of plastic materials and/or bio-plastic materials and/or biomaterials.

One advantage is to make high energy efficiency of a deodorization apparatus possible, in particular to optimize a deodorization process for deodorizing plastic materials and/or bio-plastic material and/or biomaterials.

Such objects and advantages, and still others, are achieved by a device and/or an apparatus and/or a method according to one or more of the claims set out below.

In one embodiment, a monitoring device comprises: a monitoring chamber containing one or more olfactory sensors; a first pump configured to convey a first gas along a first duct to the monitoring chamber; a valve configured to connect selectively a second duct to a source of a second gas or to a source of a third gas; and a second pump for conveying the gas selected by the valve along the second duct to the monitoring chamber, so that the monitoring chamber may be supplied selectively with the first gas, or with the second gas, or with the third gas.

The first gas may comprise, in particular, a process gas used in a deodorization process for deodorizing incoherent plastics. The first gas may be, in particular, the gas to be analyzed with the olfactory sensor. The second gas may comprise, in particular, a base gas (usable as a reference gas in measuring odour of the first gas, for example ambient air or zero air). The third gas may comprise, in particular, a cleaning gas, for example purified ambient air.

It is in particular possible to use one or more olfactory sensors, each of which comprises at least one semiconductor cell the electrical resistance variation of which may be correlated with the variation in the intensity of the odorous emission. It is pointed out that it is not necessary to determine the chemical nature of the odorous emission, but it is sufficient, in particular, to determine the trend of the intensity of the odorous emission on the basis of the response signals of the olfactory sensor/s.

The monitoring device enables an approximate measurement to be made of the presence of volatile substances, in particular of the presence of VOC and/or TOC, in a gas flow.

The monitoring device may be associated with, or integrated into, a deodorization apparatus for deodorizing incoherent plastics.

The monitoring chamber may contain, in particular, two, or three, or four, or more than four olfactory sensors. The monitoring device may comprise, in particular, a first port arranged at one end of the first duct and configured for removable connection with a sampling line that is in turn connected to a deodorizing hopper for deodorizing incoherent plastics.

The monitoring device may comprise, in particular, two distinct ports arranged respectively at an end of the third duct and at an end of the fourth duct, or a single port shared (for example through bifurcation) by the third duct and by the fourth duct. The monitoring device may comprise, in particular, a control unit configured to control the first pump, the second pump and the valve so as to convey the selected gas to the monitoring chamber passing selectively (i) either through the first duct, (ii) or through the third duct and then through the second duct, (iii) or through the fourth duct and then through the second duct.

The monitoring chamber may comprise, in particular, a first opening (obtained in a wall that bounds the monitoring chamber) at which the first duct ends and a second opening (obtained in a wall that bounds the monitoring chamber) at which the second duct ends. Alternatively, the monitoring chamber may comprise a single opening (obtained in a wall that bounds the monitoring chamber) at which a portion of duct communicating (for example through bifurcation) with the first duct and with the second duct ends.

The monitoring device may comprise, in particular, a first storage tank arranged in the first duct and/or a second storage tank arranged in the second duct. The first storage tank may be arranged, in particular, between the first pump and the monitoring chamber. The second storage tank may be arranged, in particular, between the second pump and the monitoring chamber. Alternatively, it is possible to provide a single gas storage tank (shared by the two ducts) that communicates with a single opening of the monitoring chamber (obtained in a wall that bounds the monitoring chamber) and at which the first duct and the second duct end.

The control unit may be configured, in particular, to perform the following steps in sequence: in a first step, the first pump is stationary, the second pump is active, a first inlet way of the valve is open and a second inlet way of the valve is closed, so as to generate a flow of gas (in particular, a base gas, for example ambient air) along the third duct and then along the second duct to the monitoring chamber (in this first step, the olfactory sensors can detect the base gas); in a second step, the first pump is active and the second pump is stationary, so as to generate a flow of gas (in particular, a sample gas, for example a gas processed in the deodorizing apparatus) that is conveyed along the first duct to the monitoring chamber (in this second step, the olfactory sensors can detect the sample gas); in a third step, the first pump is stationary, the second pump is active, the second inlet way is open and the first inlet way is closed, so as to generate a flow of gas (in particular, a cleaning gas, for example purified ambient air, in particular purified by a filter arranged in the fourth duct) along the fourth duct and then along the second duct to the monitoring chamber.

The monitoring chamber may contain one or more sensors arranged for measuring one or more parameters, in particular the pressure and/or the temperature and/or the relative humidity (or another parameter indicating the humidity content) and/or the gas flow rate.

The monitoring chamber may define, in particular, a single chamber that contains one or more olfactory sensors (for example two or three or four or more than four olfactory sensors) and one or more other sensors (for example two or three or more than three other sensors).

It is possible to provide, in particular, for the monitoring chamber containing (for example inside a single chamber) two or more olfactory sensors of the same type (i.e. olfactory sensors so characterized as to detect the same substance or group of substances). This can enable a possible irregularity (fault, contamination, etc) to be detected in the olfactory sensors.

The monitoring device may comprise, in particular, pressurizing means for pressurizing the monitoring chamber (i.e. for obtaining pressure inside the monitoring chamber that is greater than the external pressure). The aforesaid pressurizing means may comprise, in particular, an appropriate configuration of a gas outlet duct from the monitoring chamber, for example suitable positioning of the outlet duct and/or suitable sizing of a passage section (with a suitable diameter) of the outlet duct so as to obtain desired pressurization of the monitoring chamber during operation of the monitoring device.

The control unit may be configured, in particular, to verify a risk of condensation formation in the monitoring chamber on the basis of the signals received from the aforesaid other sensor/s.

Using the two distinct ducts (first duct and second duct) and two respective distinct pumps (first pump and second pump) to supply the sample gas to be analyzed (with relatively high odorous substances content) distinct from the supply of base gas and cleaning gas (with very reduced or absent odorous substances content) enables the components to be kept clean that are not connected to the gas with a high content of odorous substances and also enables the transition time to be reduced that occurs between switching from one operating step to the other and which is necessary for establishing the detecting system and then ensuring reliable measuring of the data by the various sensors of the system.

### BRIEF DESCRIPTION OF THE DRAWINGS

The invention can be better understood and implemented with reference to the attached drawings that illustrate some embodiments thereof by way of non-limiting example in which:

FIG. 1 shows a diagram in vertical elevation of a deodorization apparatus that can be associated with a monitoring device made in accordance with the present invention;

FIG. 2 shows a diagram of a first embodiment of a monitoring device made in accordance with the present invention;

FIGS. 3A, 3B and 3C show schematically three different operating modes of the monitoring device of FIG. 2;

FIG. 4 shows a diagram of a second embodiment of a monitoring device made in accordance with the present invention;

FIG. 5 shows a diagram of a third embodiment of a monitoring device made in accordance with the present invention;

FIG. 6 shows a diagram of a fourth embodiment of a monitoring device made in accordance with the present invention;

FIG. 7 shows a diagram of a fifth embodiment of a monitoring device made in accordance with the present invention;

FIG. 8 shows a diagram of a monitoring chamber that is usable in a monitoring device in accordance with the present invention;

FIG. 9 is a perspective view of a monitoring device made in accordance with the present invention.

### DETAILED DESCRIPTION

With reference to the attached figures, for the sake of simplicity of exposition, analogous elements of different embodiments have been indicated by the same numbering.

With reference to the figures mentioned above, with 1 a monitoring device has been indicated above that is usable, in particular, for monitoring odorous emissions contained in a gas. The monitoring device 1 may be used, in particular, to provide real time indications of the volatile organic carbon (VOC) trend and/or total organic carbon (TOC) trend present in a gas used in an industrial process, in particular a deodorization process for deodorizing incoherent plastics obtained at least partially from recycling. The processed plastics may comprise, in particular, polyolefins, for example r_HDPE, r_PP, r_PE.

The monitoring device 1 may comprise, in particular, a support S for supporting the various components of the device. The support S may comprise, in particular, a box-shaped case. The support S can define, in particular, an inner space containing the various components of the device.

The monitoring device 1 comprises at least one monitoring chamber 2. The monitoring chamber 2 may be housed, in particular, inside the support S. The monitoring chamber 2 may contain one or more olfactory sensors 3 arranged inside the monitoring chamber 2.

The monitoring device 1 comprises at least one first duct 4 configured to convey gas to the monitoring chamber 2. The first duct 4 may be housed, in particular, inside the support S.

The monitoring device 1 comprises at least one first port 5 arranged at one end of the first duct 4. The first port 5 may be arranged, in particular, on an outer wall of the support S. The first port 5 may comprise, in particular, a connector for removably connecting a pipe.

The monitoring device 1 comprises a first pump 6 arranged for moving the gas in the first duct 4 from the first port 5 to the monitoring chamber 2. The first pump 6 may comprise, in particular, a diaphragm pump. The first pump 6 may be housed inside the support S.

The monitoring device 1 comprises a second duct 7 to convey gas to the monitoring chamber 2. The second duct 7 may be housed, in particular, inside the support S.

The monitoring device 1 comprises a second pump 8 arranged for moving gas in the second duct 7 to the monitoring chamber 2. The second pump 8 may comprise, in particular, a diaphragm pump. The second pump 8 may be housed inside the support S.

The monitoring chamber 2 may comprise, in particular, an outlet opening OUT to allow the gas to exit that has been delivered to the chamber. The outlet opening OUT may be connected, in particular, to an outlet port arranged on an outer part of the support S (for example for discharge into the environment). The outlet opening OUT may be connected to the outlet port by an outlet duct. The outlet duct may be housed, in particular, inside the support S. The outlet duct may be positioned and/or dimensioned (passage section) in order to ensure pressurization of the monitoring chamber 2.

The monitoring device 1 comprises a valve 9 with one outlet way connected to the second duct 7, a first inlet way connected to a third duct 10 (base gas duct) and a second inlet way connected to a fourth duct 11 (cleaning gas duct). The valve 9 may comprise, in particular, a solenoid valve that is controllable for switching the flow of gas (base gas duct or cleaning gas duct) sent to the monitoring chamber 2. The valve 9 may be housed inside the support S.

The monitoring device 1 may comprise, in particular, two distinct ports 12 and 13 arranged respectively at one end of the third duct 10 and at an end of the fourth duct 11. Alternatively, the monitoring device 1 may comprise a single port 12′ shared by the third duct 10 and to the fourth duct 11. The two ports 12 and 13 and the port 12′ may be arranged, in particular, on an outer wall of the support S. Each port 12, 13, and 12′ may comprise, in particular, a connector for removably connecting a pipe.

The monitoring device 1 comprises an (electronic and programmable) control unit 14 configured to control the first pump 6, the second pump 8 and the valve 9 so as to convey gas to the monitoring chamber 2 passing selectively (i) either through the first duct 4, (ii) or through the third duct 10 and then through the second duct 7, (iii) or through the fourth duct 11 and then through the second duct 7.

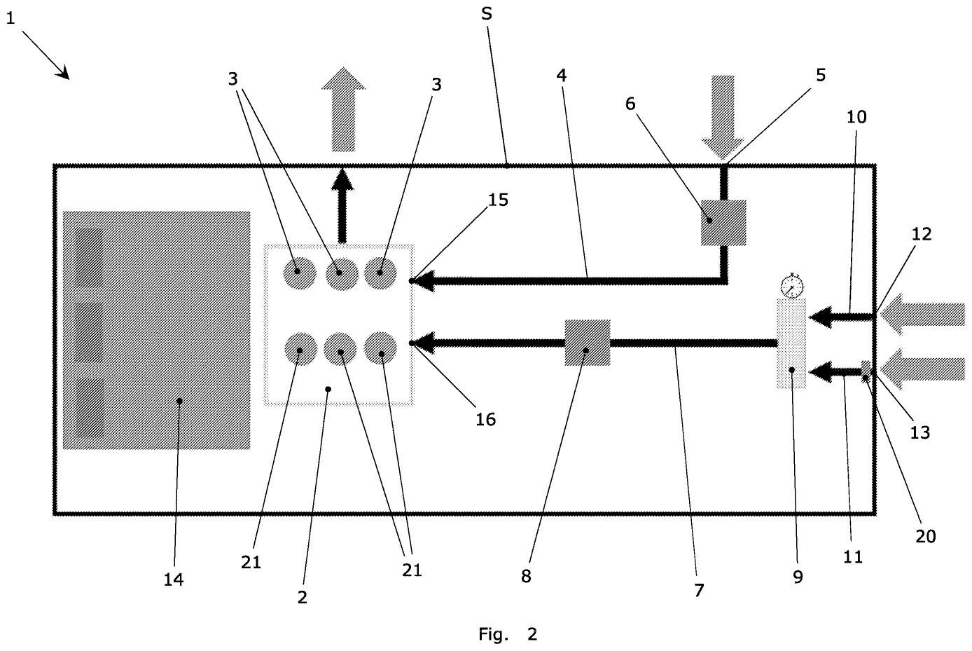

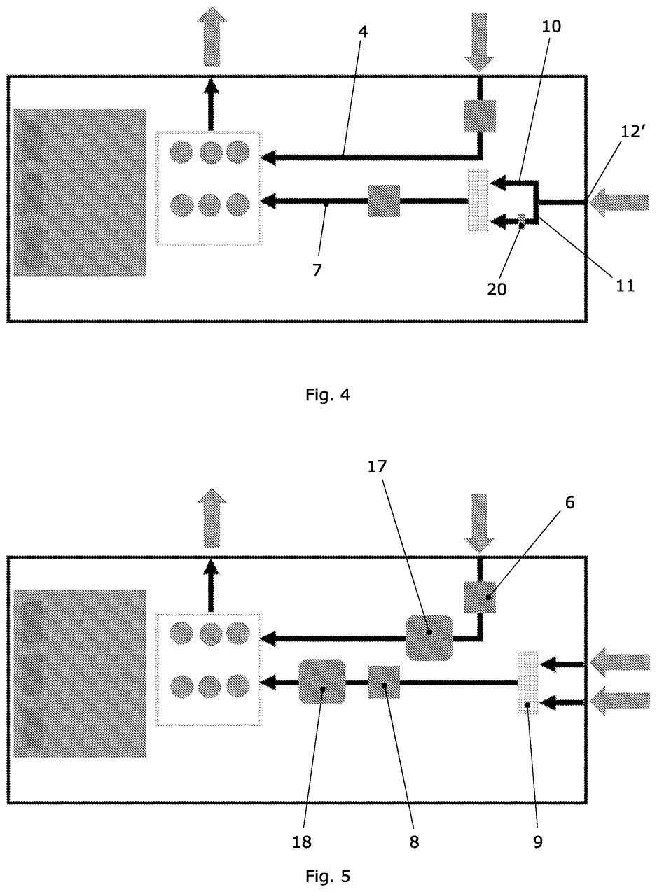

In one embodiment (see FIG. 2, or FIG. 4, or FIG. 5), the first duct 4 and the second duct 7 may be, in particular, arranged for conveying gas, respectively, to a first inlet opening 15 and to a second inlet opening 16 of the monitoring chamber 2.

In one embodiment (see FIG. 5), the monitoring device 1 may comprise, in particular, a first storage tank 17 arranged in the first duct 4 and/or a second storage tank 18 arranged in the second duct 7. The first storage tank 17 and the second storage tank 18 may be housed inside the support S.

The first storage tank 17 may be arranged, in particular, between the first pump 6 and the monitoring chamber 2. The second storage tank 18 may be arranged, in particular, between the second pump 8 and the monitoring chamber 2.

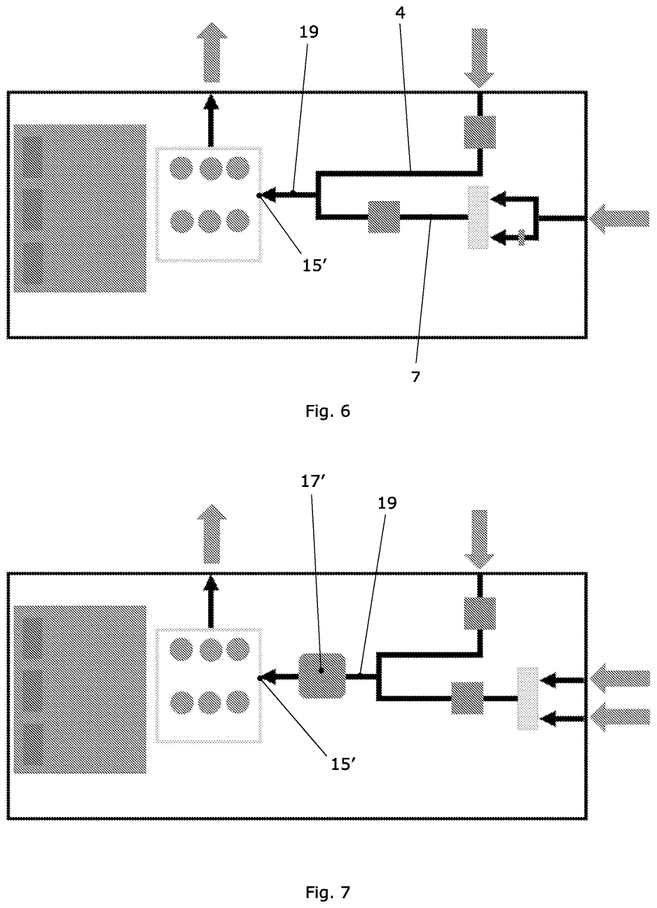

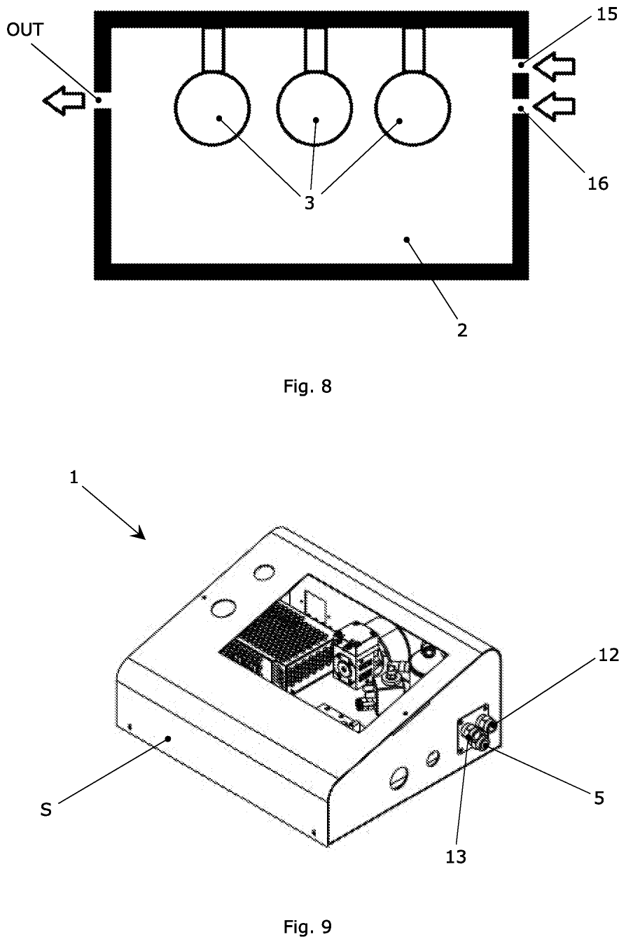

In one embodiment (see FIG. 6 or FIG. 7), the first duct 4 and the second duct 7 may, in particular, communicate (for example, through a point of confluence) with a duct section 19 arranged for conveying gas to an inlet opening 15′ of the monitoring chamber 2.

In one embodiment (see FIG. 7), the monitoring device 1 may comprise, in particular, a storage tank 17′ communicating with an opening 15′ of the monitoring chamber 2. In particular, the first duct 4 and the second duct 7 may end at the storage tank 17′.

The monitoring device 1 may comprise, in particular, a purifying filter 20 arranged for purifying a flow of gas passing through the fourth duct 11. The purifying filter 20 may comprise, in particular, an adsorption filter, for example an activated carbon filter. The purifying filter 20 may be arranged, in particular, inside the support S. The purifying filter 20 may be arranged, in particular, at the connecting port 13 connecting a pipe supplying ambient air to be purified. The purifying filter 20 may be arranged, in particular, in the pipe supplying ambient air to be purified.

The control unit 14 may comprise, in particular, a main electronic board and a sensor electronic board.

The control unit 14 may be configured, in particular, to perform the following steps in sequence:
a first step in which the first pump 6 is stationary, the second pump 8 is active for moving gas, the valve 9 is so commanded that the second inlet way is closed, and the first inlet way is open, with the possibility of generating a flow of a base gas, in particular ambient air, in the third duct 10 and then in the second duct 7 to the monitoring chamber 2;
a second step in which the first pump 6 is active and the second pump 8 is stationary, so that a flow of a sample gas can be generated, in particular a gas processed in a deodorization apparatus, in the first duct 4 to the monitoring chamber 2; and
a third step in which the first pump is stationary, the second pump 8 is active, the valve 9 is so commanded that the second inlet way is open and the first inlet way is closed, so that it is possible to generate a flow of a cleaning gas, in particular purified ambient air, in the fourth duct 11 and then in the second duct 7 to the monitoring chamber 2.

In the first step the aforesaid one or more olfactory sensors 3 measure at least one base value (indicating odorous emissions) of the base gas. In the second step, the aforesaid one or more olfactory sensors 3 measure at least one first value or analysis value (indicating odorous emissions) of the sample gas. The first value or analysis value is used, together with the base value that constitutes a reference value, to determine a level of odorous emissions of the sample gas. The aforesaid one or more olfactory sensors 3 can measure, in fact, a plurality of values continuously both for the base gas and for the sample gas. The sample gas may be monitored for a prolonged period over the course of the deodorization process.

The monitoring chamber 2 may comprise, in particular, one or more sensors 21 arranged for measuring one or more parameters in the monitoring chamber 2. The control unit 14 may be configured, in particular, to receive signals from the aforesaid one or more sensors 21 and to verify, on the basis of such signals, a risk of formation of condensation in the monitoring chamber 2. The aforesaid one or more parameters measured by the sensors 21 may include, in particular, the pressure and/or the temperature and/or the relative humidity (or another parameter indicating the humidity content) and/or the gas flow rate in the monitoring chamber 2. In the specific embodiments disclosed here, the monitoring chamber 2 comprises the sensors for detecting all four aforesaid parameters. It is nevertheless possible to provide in the monitoring chamber 2 the sensors that are suitable for detecting a lower number of parameters, for example only pressure, temperature and relative humidity, or yet other combinations.

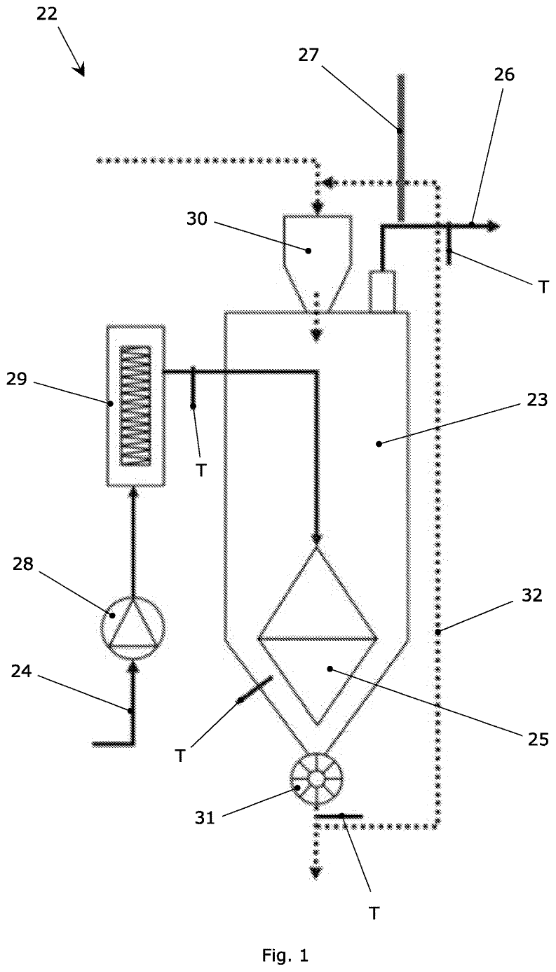

The monitoring device 1 may be used, in particular, in a deodorization apparatus 22 (see FIG. 1) comprising a hopper 23 for containing incoherent plastics to be deodorized. The deodorizing apparatus 22 comprises an inlet line 24 for delivering the process gas into the hopper 23. The inlet line 24 may end with a diffuser cone 25 arranged inside the hopper 23. The deodorizing apparatus 22 comprises an outlet line 26 for extracting process gas from the hopper 23. The monitoring device 1 may be integrated into or associated with or connected to the deodorizing apparatus 22.

The deodorizing apparatus 22 comprises a sampling line or monitoring line 27 arranged for detecting process gas used (sample gas to be analyzed by the olfactory sensor/s 3) in the deodorization process. In the specific embodiment, the sampling or monitoring line 27 is arranged for detecting the gas from the outlet line 26, although it is possible to provide other sampling points, for example inside the hopper 23 near or at the height of the diffuser cone 25, or in still other points (for example above the diffuser cone 25). The monitoring line 27 is connected to the first port 5 of the monitoring device 1.

The deodorizing apparatus 22 may comprise, in particular, an actuator 28 configured to move the process gas. The actuator 28 may comprise, in particular, a blower. The actuator 28 may be configured, in particular, to suck air from the environment (for example, filtered air). The deodorizing apparatus 22 may comprise, in particular, a heating device 29 (for example of the resistance type) for heating the process gas before delivering the process gas to the hopper 23. The heating device 29 may be thermostated.

The deodorizing apparatus 22 may comprise, in particular, a loading zone 30 of the material to be deodorized (for example, at the top of the hopper 23) and a discharge zone 31 of the deodorized material (for example, on the bottom of the hopper 23). The loading zone 30 may comprise a loading hopper and the discharge zone 31 may comprise a rotating device for controlled continuous discharging of the material.

The deodorizing apparatus 22 may comprise, in particular, temperature sensors T for detecting the temperature of the process gas and/or of the material, for example a sensor T in the inlet line 24, a sensor T in the outlet line 26, a sensor T in the discharge zone 31, one or more sensors T inside the hopper 23.

It is possible to provide a recirculating line 32 of the material that enables a part of the processed material to be recirculated from the discharge zone 31 of the material to the loading zone 30 of the material to be subjected again to deodorization in the hopper 23. This recirculation of the material occurs in a controlled manner controlled by a movement device for moving the material (not shown).

The deodorizing apparatus 22 may be of the open circuit type, as in the embodiment disclosed here, in which the process gas is air taken from the environment, or the closed-circuit type, in which the process gas is taken from the outlet of the hopper 23 and then processed to be returned to the hopper.

In the closed-circuit configuration, it is possible to arrange an odour reduction and/or absorption system in the circuit, for example a system comprising an activated carbon absorption filter.

As said, the first port 5 del monitoring device 1 may be, in particular, connected to the outlet line 26 by a monitoring line 27 that enables the sample gas to be sampled, in the specific case the process gas used exiting the hopper 23. The first port 5 of the monitoring device 1 may be, in particular, connected directly to the hopper 23 by a monitoring line that enables the sample gas to be sampled, for example, from the diffusion zone of the process gas near the diffuser cone 25.

The sampling point of the sample gas may be determined, in particular, as a function of the type of circuit (open or closed) and/or in function of the number of monitoring devices installed in the deodorizing apparatus.

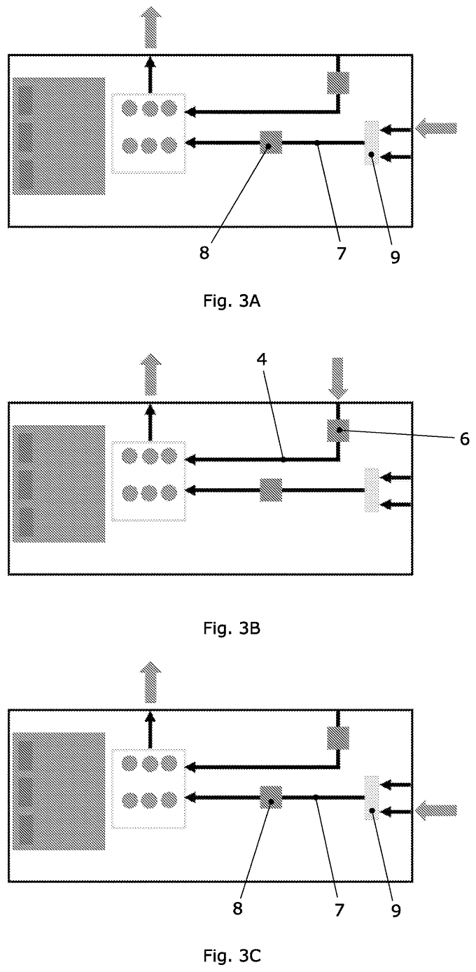

Operating the monitoring device 1 may actuate, in particular, a method for monitoring a deodorization process for deodorizing incoherent plastics. The method comprises a preliminary step of providing the monitoring device 1 and a step (first step, see FIG. 3A) of generating a flow of a base gas, in particular ambient air, in the third duct 10 and then in the second duct 7 to the monitoring chamber 2 and then measuring at least one base value of the base gas by the aforesaid one or more olfactory sensors 3.

The method comprises a step (second step, see FIG. 3B) of generating a flow of a process gas used in the deodorization process (sample gas) through the first duct 4 to the monitoring chamber 2, to measure at least one first value, or actual analysis value of the process gas used by the aforesaid one or more olfactory sensors 3 and then to use the first value and the base value to determine an approximate value of the odorous emission in the sample gas sampled by the deodorization process.

The method comprises a step (third step, see FIG. 3C) of generating a flow of a cleaning gas, in particular purified ambient air, along the fourth duct 11 and then the second duct 7 to the monitoring chamber 2.

In the specific embodiments, the monitoring chamber 2 comprises three olfactory sensors 3 (although a different greater or lesser number of olfactory sensors is possible) characterized differently from one another, where the different characterization enables specific VOC substances or groups of substances to be detected. In other words, each olfactory sensor 3 has been chosen and made with specific characteristics that differ from sensor to sensor, in function of the nature of a plurality of VOC substances the presence of which it is intended to detect.

The control unit 14 may comprise control FW and/or control SW of the monitoring device 1. The main board may comprise, in particular, a supply input, for example a 24V supply input, an output for controlling the valve 9 (in particular for switching the steps of detecting the base gas and cleaning gas of the monitoring chamber 2 after a given period of use), an output for controlling the first pump 6, an output for controlling the second pump 8, an ETH port for exchanging data, a USB port for updating the SW, one or more connectors for connecting the various sensors 21 arranged in the monitoring chamber 2. The main board may be connected to the sensors board (that governs the olfactory sensors 3), for example, by a 20-pin cable or other type of cable.

The main board acquires the signals of the odorous emissions received from the olfactory sensors 3. These signals are processed (for example in the PLC of the deodorizing apparatus 22) to obtain a numeric entry that is proportional to the intensity of the odorous emissions and then correlatable with the presence of odorous substances. In particular, each olfactory sensor 3 emits voltage or electric resistance signals and the significant parameter that is processed by the control unit 14 or by the PLC of the deodorizing apparatus 22 relates to the variation of the voltage or electric resistance signal for each olfactory sensor 3. Determining the odorous emissions comprises, in particular, calculating a value equal to a quotient between a numeric value measured for the base gas (used as a reference value) and a numeric value measured for the sample gas.

The aforesaid first step of the monitoring method, in which in the monitoring chamber 2 the base gas is detected, in particular the ambient air, is used to define a base value, in particular an electric resistance base value. This first step may last, for example, for a set time, for example 15 minutes, in order to acquire the base value for each olfactory sensor 3.

The second step, i.e. the actual analysis step, in which the gas used taken from the hopper 23 or from the outlet line 26 is directed to the monitoring chamber 2, may also have a relatively long set duration, for example of 60 or 120 minutes or even longer (duration that may depend in certain cases also on the duration of the deodorization process).

Processing signals comprises the calculating step (in particular, for each olfactory sensor 3) a ratio (i.e. a numeric quotient) between the electric resistance, produced by the contact with the odorous molecules of the sample gas, and the initial reference electric resistance of the base gas (i.e. of the ambient air not affected by the deodorization process).

The third cleaning step, which is used to clean the olfactory sensors 3 and the sensors 21, with resulting restoration of the initial configuration, may have a duration that is proportional to the duration of the second analysis step of analyzing the process gas used.

The valve 9 (like pumps 6 and 8) may be controlled by a timer connected to the control unit 14.

The deodorization process may run with incoherent plastics delivered to the hopper 23 in a continuous flow or in batches. The process gas used may be monitored in a programmed cycle in which the three steps alternate: base (or initial reference), analysis and cleaning. The odorous emissions of the gas in the base and analysis steps may be measured continuously.

The monitoring device 1 may be provided in an embodiment that is integrated with the deodorizing apparatus 22, in which case the monitoring device 1 is connected to the control unit of the apparatus, for example with the PLC and the man-machine interface of the apparatus. A stand-alone embodiment is possible that is provided with its own man-machine interface, for example for experimental use.

1-12. (canceled)

13. A monitoring device, comprising a monitoring chamber, one or more olfactory sensors arranged in said monitoring chamber, a first duct for conveying gas towards said monitoring chamber, a first port arranged at one end of said first duct, a first pump arranged to move gas in said first duct from said first port towards said monitoring chamber, a second duct for conveying gas to said monitoring chamber, a valve with one outlet way connected to said second duct, and a first inlet way connected to a third duct, a second pump arranged to move gas in said second duct towards said monitoring chamber, a second inlet way connected to a fourth duct, two distinct ports arranged respectively at one end of said third duct and at one end of said fourth duct or a common port connected to said third duct and to said fourth duct, and a control unit configured to control said first pump, said second pump and said valve so as to convey gas towards said monitoring chamber by selectively passing (i) or through said first duct, (ii) or through said third duct and then through said second duct, (iii) or through said fourth duct and then through said second duct.

14. The device according to claim 13, wherein said first duct and said second duct are arranged for conveying gas, respectively, to a first opening and to a second opening of said monitoring chamber.

15. The device according to claim 13, comprising a first storage tank arranged in said first duct.

16. The device according to claim 15, wherein said first storage tank is arranged between said first pump and said monitoring chamber.

17. The device according to claim 15, comprising a second storage tank arranged in said second duct.

18. The device according to claim 17, wherein said second storage tank is arranged between said second pump and said monitoring chamber.

19. The device according to claim 13, comprising a second storage tank arranged in said second duct.

20. The device according to claim 19, wherein said second storage tank is arranged between said second pump and said monitoring chamber.

21. The device according to claim 13, wherein said first duct and said second duct communicate with a duct section arranged to convey gas to an opening of said monitoring chamber.

22. The device according to claim 13, comprising a storage tank communicating with an opening of said monitoring chamber and at which said first duct and said second duct end.

23. The device according to claim 22, comprising a storage tank communicating with an opening of said monitoring chamber and at which said first duct and said second duct end.

24. The device according to claim 13, wherein said first pump comprises a diaphragm pump and/or in which said second pump comprises a diaphragm pump.

25. The device according to claim 13, comprising a purifying filter arranged for purifying a gas flow passing through said fourth duct; said purifying filter comprising an adsorption filter.

26. The device according to claim 13, wherein said control unit is configured to perform the following steps in sequence: a first step in which the first pump is stationary, the second pump is active, the second inlet way is closed and the first inlet way is open to allow a flow of a base gas to be generated in said third duct and in said second duct towards said monitoring chamber, said one or more olfactory sensors measuring at least one base value of said base gas; a second step in which the first pump is active and the second pump is stationary to allow a flow of a sample gas to be generated in said first duct towards said monitoring chamber, said one or more olfactory sensors measuring at least one first value of said sample gas, said first value and said base value being usable for determining the odorous emissions in the sample gas; a third step in which the first pump is stationary, the second pump is active, the second inlet way is open and the first inlet way is closed to allow a flow of a cleaning gas to be generated in said fourth duct and in said second duct towards said monitoring chamber.

27. The device according to claim 13, comprising one or more sensors arranged for measuring one or more parameters in said monitoring chamber, said control unit being configured to receive signals from said one or more sensors and to check a risk of formation of condensation in said monitoring chamber on the basis of said signals; said one or more parameters including the pressure and/or the temperature and/or the relative humidity and/or the gas flow rate in said monitoring chamber.

28. A deodorizing apparatus comprising a hopper for containing incoherent material to be deodorized, an inlet line for delivering a process gas to said hopper, an outlet line for extracting process gas from said hopper, at least one olfactory sensor arranged for monitoring the process gas, wherein said at least one olfactory sensor belongs to a monitoring device made according to claim 13 and used for monitoring process gas used; said first port of said monitoring device being connected to said hopper or said outlet line by a monitoring line.

29. A method for monitoring a deodorization process of incoherent material, in particular incoherent plastics and/or incoherent biomaterial, said method comprising the steps of:
providing a monitoring device made according to claim 13;
generating a flow of a base gas in said third duct and in said second duct towards said monitoring chamber and measuring at least one base value of said base gas by said one or more olfactory sensors;
generating a flow of a process gas used in said deodorization process in said first duct towards said monitoring chamber, measuring at least one first value of said process gas used by said one or more olfactory sensors and using said first value and said base value to determine a value indicating odorous emissions contained in said process gas used;
generating a flow of a cleaning gas in said fourth duct and in said second duct towards said monitoring chamber.
